(19) United States
(12) Patent Application Publication
(10) Pub. No.: US 2024/0260853 A1
(43) Pub. Date: Aug. 8, 2024

(54) HEAD MOTION DETECTION AND CORRECTION IN MRI USING A PRESSURE SENSING PAD

(71) Applicants: Vanderbilt University, Nashville, TN (US); University of Arkansas, Fayetteville, AR (US)

(72) Inventors: Saikat Tarun Sengupta, Nashville, TN (US); Yue Chen, Fayetteville, AR (US); Mishek Musa, Fayetteville, AR (US)

(21) Appl. No.: 18/163,379

(22) Filed: Feb. 2, 2023

Related U.S. Application Data
(60) Provisional application No. 63/306,067, filed on Feb. 2, 2022.

(51) Int. Cl. A61B 5/11; A61B 5/00

(52) U.S. Cl. CPC A61B 5/1114; A61B 5/6835; A61B 5/6892; A61B 5/70

(57) ABSTRACT

This disclosure is directed to systems and methods for detecting and estimating motion of a head of a patient in magnetic resonance imaging (MRI). The system includes a pad for supporting a patient's head. The pad has protrusions and each of the protrusions define a chamber filed with a fluid. The system includes pressure sensors and each of the pressure sensors are operatively connected with a respective chamber. Each of the pressure sensors can sense a pressure within each of the respective chambers. The system includes a controller. The controller can receive data indicative of the pressure within each of the respective chambers, and can detect the motion of the head of the patient based on the data indicative of the pressure within each of the respective chambers.

### CROSS-REFERENCE TO RELATED APPLICATION

This application claims the benefit of U.S. Provisional Application No. 63/306,067 filed Feb. 2, 2022, the disclosure of which is incorporated by reference in its entirety.

### FIELD OF THE INVENTION

The present disclosure generally relates to head motion detection and correction for scanning machines, and more particularly, to head motion detection and correction systems and methods that utilize a pressure sensing pad.

### BACKGROUND

Head motion is a persistent problem in magnetic resonance imaging (MRI) that can adversely impact brain imaging in the clinic and in the research laboratory. Head motion can generate data corruption such as blurring, ghosting, signal intensity variations and SNR loss in images. This data corruption can reduce diagnostic quality and can introduce errors in any parameters estimated from the corrupted data. Head motion issues can be particularly acute in pediatric and older patients who find it difficult to hold still for long scan and exam durations.

Head motion in an MRI scanner can be defined by 6 Degrees of Freedom (6DoF) rigid body parameters consisting of 3 rotations about the three axes and 3 translations. Based on this model, a number of techniques have been developed to measure and correct head motion. In spite of this, motion correction is not routinely employed either in the clinic or in the research setting.

Current correction methods include FID/1D/2D/3D water navigators, fat navigators, optical systems with and without markers, NMR markers, Wi-Fi markers, volume registrations, self-navigated sequences and more recent wireless ‘pilot-tone’ signal, incoherent encoding and deep learning-based methods. Every method suffers from drawbacks that have prevented wide-scale adoption. For example, navigators alter the MRI sequence, impact magnetization history and extend scan times. Optical methods require either markers on the head, glasses or bite bar and open head coils with a direct line of sight. Volume registration-based methods extend scan times. NMR and Wi-Fi marker methods also require markers on the head and patient compliance. Newer methods based on disordered encoding trajectories and deep learning are promising, but have only been shown for select sequences and in the retrospective setting, which does not correct for through plane motions in multi-slice imaging.

There thus exists a need for a motion detection and correction method that a) does not interfere with the MRI sequence, require a particular k-space trajectory or lengthen the scan time (i.e. uses a non-NMR signal-based motion detection), b) can be used prospectively (i.e., motion can be estimated in real time and estimates fed back to the sequence rapidly for geometry update before the next imaging shot), c) does not require markers that add to patient discomfort (i.e. patient would be oblivious to the presence of the sensor), d) works for any MRI sequence, e) does not rely on line of sight through the head coil (i.e. works for any coil design) and f) does not require extensive calibration inside the scanner.

### SUMMARY

The foregoing needs are met by the systems and methods of this disclosure. According to one aspect of the present disclosure, a system for estimating a motion of a head of a patient in a scanner includes a pad. The pad is configured to support the head of the patient within the scanner. The pad may include a plurality of protrusions that protrude from a surface of the pad. Each of the plurality of protrusions define a chamber filed with a fluid. The system also includes a plurality of pressure sensors. Each of the plurality of pressure sensors are operatively connected with a respective chamber of the plurality of protrusions, and each of the plurality of pressure sensors are configured to sense a pressure within each of the respective chambers and to generate data indicative of the pressure. The system also includes a controller that is configured to: receive the data indicative of the pressure within each of the respective chambers, and detect the motion of the head of the patient based on the data indicative of the pressure within each of the respective chambers.

Implementations may include one or more of the following features. When the controller detects the motion of the head of the patient during a scan of the patient by the scanner the controller is configured to at least one of estimate the motion of the head of the patient, automatically stop the scanner, signal that the motion has been detected, estimate a quantity of data corrupted by the motion, estimate a quantity of data corrupted by the motion and signal the quantity, or provide feedback to patient. Each of the plurality of protrusions define a convex shape. The fluid is a gas. The fluid is a liquid. The system may include a plurality of fluid pathways that fluidly connect each respective chamber of the plurality of protrusions with the respective pressure sensor of the plurality of pressure sensors. The plurality of fluid pathways each include a segment that is integral with the pad. The scanner is an MRI scanner and the system may include the MRI scanner configured to scan the head of the patient. The controller is further configured to correct a scan of the head of the patient from the MRI scanner based on the estimate of the motion of the head. The controller is configured to correct the scan of the head of the patient from the MRI scanner after completion of the scan. The controller is configured to estimate the motion of the head based on the data indicative of the pressure within each of the respective chambers and correct the scan of the head of the patient during the scan. The plurality of pressure sensors are provided outside of an interior of the MRI scanner. The MRI scanner may include an MRI head coil. The MRI head coil defines a space within an interior of the MRI head coil, and the space is configured to receive the pad and the head of the patient. The plurality of pressure sensors are MRI compatible.

According to another aspect of the present disclosure, a method for correcting for motion of a body part in an scanner includes arranging a pad in the scanner. The method also includes sensing, via a plurality of sensors operatively connected to the pad, a pressure applied by the body part to the pad. The method also includes detecting a motion of the body part based on the data. The method also includes acting in response to the detecting of the motion of the body part.

Implementations may include one or more of the following features. Acting in response to the detecting of the motion of the body part comprises at least one of estimating the motion of the head of the patient, automatically stopping the scanner, signaling that the motion has been detected, estimating a quantity of data corrupted by the motion, estimating a quantity of data corrupted by the motion and signaling the quantity, or providing feedback to patient. The detecting the motion is performed during a scan of the body part by the scanner. The body part is a head of a patient. The sensing of the pressure applied by the body part to the pad may include sensing a pressure of a fluid partially contained within the pad. The scanner is an MRI scanner and the plurality of sensors are MRI compatible.

There has thus been outlined certain embodiments of the present disclosure in order that the detailed description thereof herein may be better understood, and in order that the present contribution to the art may be better appreciated. There are additional embodiments of the present disclosure that will be described below, and which form the subject matter of the claims appended hereto.

In that respect, before explaining at least one aspect of the present disclosure in detail, it is to be understood that the embodiments described herein are not limited in their application to the details of construction and to the arrangements of the components set forth in the following description or illustrated in the drawings. The systems and methods of this disclosure are capable of aspects in addition to those described, and of being practiced and carried out in various ways.

### BRIEF DESCRIPTION OF THE DRAWINGS

In order that the present disclosure may be readily understood, aspects are illustrated by way of examples in the accompanying drawings, in which like parts are referred to with like reference numerals throughout.

FIG. 1 shows a schematic view of a system for detecting and estimating motion of a head of a patient in a magnetic resonance imaging scanner.

FIG. 2 shows a perspective view of a pad for sensing pressure exerted by the head of the patient in the magnetic resonance imaging scanner.

FIG. 3 shows a cross section view of the pad of FIG. 2.

FIG. 4 shows a perspective view of an embodiment of a calibration of the system outside of the MRI scanner.

FIG. 5 shows the results of a motion estimation using a calibrated sensing pad when a skull phantom stays on the calibrated sensing pad.

FIG. 6 shows the results of motion estimation using a calibrated sensing pad when the skull phantom is lifted off the calibrated sensing pad and put back down on the calibrated sensing pad.

FIG. 7 shows a process for detecting and correcting motion of a body part in an MRI scanner.

### DETAILED DESCRIPTION

This disclosure is directed to systems and methods for using pressure sensors to detect, estimate, and correct for head motion during MRI scans. An optimized array of pressure sensors can be integrated into a pad and can be used to detect and estimate head motions by continuously monitoring pressure changes occurring due to head movements during a scan. The pressure sensors can be any MRI compatible sensor, including for example, optical or pneumatic pressure sensors. In embodiments, the pressure sensors are pneumatic sensors which can reduce costs and improve scalability.

The pressure sensors can be operatively connected to the pad, and the pad can replace the standard head cushion used in MRI scans and that can record head motion related pressure changes in real time. The systems and methods can undergo a rapid subject-specific calibration performed either outside or inside the scanner. Six degree of freedom (6DoF) head motions can be estimated during the scan and motion corrected images can be reconstructed retrospectively. In embodiments, the system and methods can perform either prospective real time motion correction or retrospective correction in which motion corrupted images can be corrected after the scan using motion data recorded by the pad.

The pad can be formed of a soft silicone (or other pliable or deformable composition such as rubber, or various flexible 3D printing resins such as Flexible 80A Resin). The pad can contain and/or be operatively connected to a matrix of fluid pressure sensors that are sensitive to the pressure imparted by a moving head inside the scanner. By measuring the pressure changes created by the moving head in the individual sensor cells in real-time, the motion of the head can be tracked during an MRI scan. The system can include the pad, fluid tubes, electronic pressure sensors, and microprocessor-based data acquisition and transfer.

The systems and methods of this disclosure can allow for 6 DoF head motion estimation. The systems and methods of this disclosure are advantageous because they 1) do not interfere with the MRI sequence or signal; 2) do not lengthen the MRI sequence; 3) work in any MRI scanner; 4) work for any head coil; 5) work for any MRI sequence; 6) do not need makers on the subject; 7) do not require optical cameras and direct line of sight; 8) are subject friendly; 9) can perform real time tracking; 10) can be used for prospective motion correction (e.g., can be employed for ‘real time’ motion correction in which the pressure signals can be fed to an MRI scanner's respiratory triggering mechanism to enable real-time motion correction); and 11) can be manufactured at low cost. The system can also simply be used for motion ‘detection’ in which motion of the head during an MRI scan is detected, allowing for the operator to stop immediately the scan, thereby saving time and money. In this mode, the system can provide continuous motion monitoring during any scan.

The systems and methods of this disclosure can therefore be used to reduce motion related image degradation in all brain MRI scans. The systems and methods of this disclosure can also be used in other modalities such as CT, PET, and SPECT, as well as in radiation therapy of brain tumors. Accordingly, although embodiments of this disclosure may use the term “MRI scanner” or the like it is to be understood that the embodiments can also include other scanning modalities.

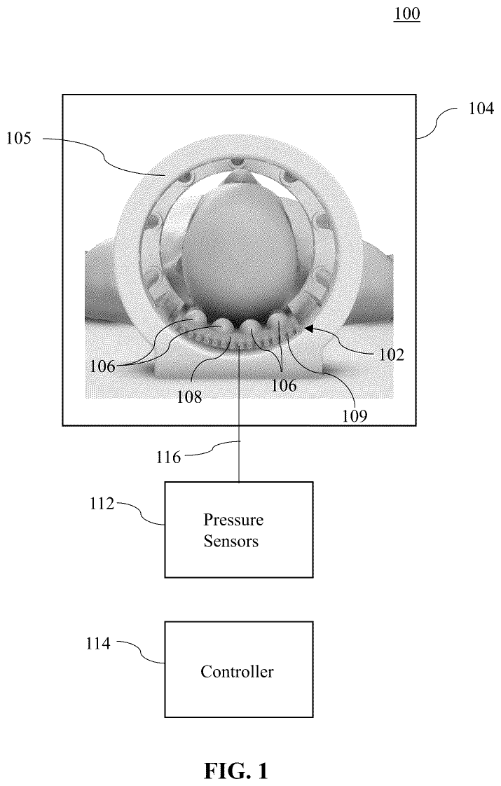

These and other aspects are described further below in the descriptions of FIGS. 1-7. It is to be understood that the figures and descriptions of the present invention may have been simplified to illustrate elements that are relevant for a clear understanding of the present invention, while eliminating, for purposes of clarity, other elements such as elements found in a typical MR system, typical MRI motion correction system, typical method of using an MR system, or typical method of correcting motion in an MRI. Those of ordinary skill in the art will recognize that other elements may be desirable and/or required in order to implement the present invention. However, because such elements are well known in the art, and because they do not facilitate a better understanding of the present invention, a discussion of such elements is not provided herein. It is also to be understood that the drawings included herewith only provide diagrammatic representations of the presently preferred structures of the present invention and that structures falling within the scope of the present invention may include structures different than those shown in the drawings.

FIG. 1 shows a schematic view of a system 100 for estimating a motion of a head of a patient in magnetic resonance imaging. The system 100 includes a pad 102. The pad 102 can support the head of the patient within an MRI scanner 104 that can scan the head of the patient. In embodiments, the pad 102 can support other body parts such as hands, feet, arms, legs, or an entirety of the body. For example, the pad 102 can support the head of the patient within an MRI head coil 105 (e.g., a 3 Tesla 32 channel head coil). of the MRI scanner 104. The MRI head coil 105 can define a space within an interior of the MRI head coil 105 and the space can receive the pad 102 and the head of the patient. In embodiments, the system 100 can include the MRI scanner 104.

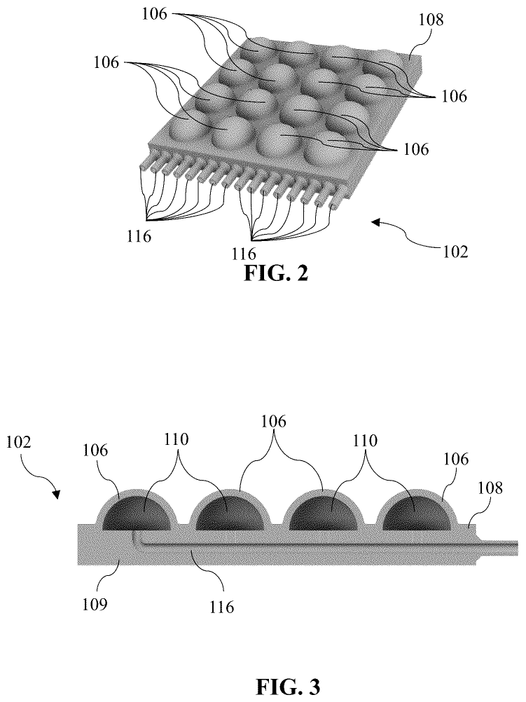

FIGS. 2 and 3 respectively show a perspective and cross section view of the pad 102. The pad 102 can comprise a plurality of protrusions 106. The plurality of protrusions 106 can be soft. The plurality of protrusions 106 can protrude from a surface 108 of the pad 102. The surface 108 can be a top surface that faces the patient when the pad 102 is within the MRI scanner 104. The pad 102 can include another surface 109. The surface 109 can be a bottom surface that opposes the surface 108 and that is configured to be supported by the MRI head coil 105. Each of the plurality of protrusions 106 can define a convex shape. Each of the plurality of protrusions 106 can define a chamber 110. The chamber 110 can be filled with a fluid. In embodiments, the fluid can be a gas such as for example air. In embodiments, the fluid can be a liquid that does not produce an MRI signal such as for example Deuterium Oxide.

In embodiments, the pad 102 can be formed of a pliable material such as for example silicone, rubber, etc. The pad 102 can be formed in multiple distinct pieces such as first and second bodies that are subsequently joined to seal the chambers 110 and the fluid pathways 116 (described further subsequently herein). In embodiments, the pad 102 can be formed from a unitary body.

The system 100 can include a plurality of pressure sensors 112. Each of the plurality of pressure sensors 112 can be operatively connected with the respective chamber 110 of the plurality of protrusions 106. For example, the pad 102 can include a plurality of fluid pathways 116 (e.g., chambers, tubes, pipes, supply lines, etc.) for each of the plurality of protrusions 106. The plurality of fluid pathways 116 can be individually fluidly connected to each chamber 110 with a respective pressure sensor 112. In embodiments, at least part of the fluid pathways 116 can be integrally formed in the pad 102. Additionally or alternatively, the fluid pathways 116 can include distinct segments (e.g., chambers, tubes, pipes, supply lines, etc.). Each of the plurality of pressure sensors 112 can sense the pressure within each of the respective chambers 110. For example, the pressure of the fluid filling each chamber 110 can be sensed by the plurality of sensors 112 in real time and the pressure can increase or decrease in response to movement of the head of a patient interacting with (e.g., compressing or releasing compression) any of the protrusions 106. Each of the plurality of sensors 112 can generate data indicative of the pressure during the sensing.

The system 100 can include a controller 114. The controller 114 can be operatively connected to each of the plurality of pressure sensors 112. The controller 114 can receive the data indicative of the pressure within each of the respective chambers 110 from each of the plurality of pressure sensors 112. The controller 114 can estimate the motion of the head of the patient based on the data indicative of the pressure within each of the respective chambers 110 received from each of the plurality of pressure sensors 112. That can be accomplished without MRI sequence navigators, head markers, or cameras, although such devices can be used to calibrate the system 100. In embodiments, the controller 114 can estimate the motions required to correct a scan of the head of the patient from the MRI scanner 104. In embodiments, the controller 114 can correct a scan of the head of the patient from the MRI scanner 104 based on the estimate of the motion of the head by supplying the motion data to the MRI scanner 104 in real time. In embodiments, the controller 114 can correct a scan of the head of the patient from the MRI scanner 104 based on the estimate of the motion of the head after completion of the scan. The controller 114 can also be used to detect motion corrupted data and guide either scan abortion or provide feedback to the patient for improved motion control.

The controller 114 can include a microcontroller dedicated to the head motion detection. For example, the micro controller can include a max ADC sampling rate of ˜9600 Hz or ˜0.1 ms. In such embodiments, the controller 114 can include an external controller that can receive the head motion data and process it for scan correction. Alternatively, a single controller 114 can both estimate head motion and correct the scan.

In embodiments, the pressure sensors 112 and the controller 114 can each be disposed outside of the interior of the MRI scanner 104, which can reduce or eliminate any scan interference that can be caused by the pressure sensors 112 and/or the controller 114 and can protect the pressure sensors 112 and the controller 114 from RF signals generated by the MRI scanner 104. In embodiments, the pressure sensors 112 and/or the controller 114 can each have RF shielding to the pressure sensors 112 and/or the controller 114 from RF signals generated by the MRI scanner 104. For example, each of the pressure sensors 112 and/or the controller 114 can be disposed in a box that shields the pressure sensors 112 and/or the controller 114 from RF signals.

In embodiments, the controller 114 can detect the motion of the head of the patient based on the date indicative of the pressure within each of the respective chambers 110 receive from each of the plurality of pressure sensors 112. In embodiments, when the controller 114 detects the motion of the head of the patient during a scan of the patient by the MRI scanner 104 the controller can perform any of the following actions: estimate the motion of the head of the patient; automatically stop the MRI scanner 104, signal that the motion has been detected (e.g., by emitting an audible alert, displaying a message, etc.), estimate a quantity of data corrupted by the motion, estimate a quantity of data corrupted by the motion and signal the quantity (e.g., by displaying a message), or provide feedback to patient. Estimating for an operator an amount of motion corrupted data and an amount of motion-free data collected during a MRI can be help an operator determine if the motion corrupted data compromised the MRI. Providing feedback (e.g., in real time) to the patient using (e.g., using a screen, earphone, or functional equivalent) can provide an opportunity for a patient to self-correct the motion and prevent the scan from needing to be repeated.

In embodiments, the controller 114 can estimate the motion of the head of the patient based on the data indicative of the pressure within each of the respective chambers 110 received from each of the plurality of pressure sensors 112 using a higher order least squares formalism. For example, the head rotation angle around an MRI scanner's z-axis Oz at each time frame can be determined based on the pressure measurement q (received from the respective pressure sensor 112) at that time point, at a corresponding location of the measurement cell s on the pad 102, where s ranges from 1 to 16. A system calibration coefficient A₁ can map them together using the following higher order polynomial equation:

$$θ z ( s , q ) = ψ {( s )}^{T} {A}_{1} γ ( q ) \qquad ( 1 )$$

where ψ(s)=[1,s,s², . . . , s^{v-1}]^{T} and γ(q)=[1,q,q², . . . , q^{w-1}]^{T} are the modal representations of the input variables s and q. v and w are the maximum order of the polynomials needed to estimate the head rotation.

To find the matrix A₁ in a least square sense, a set of calibration measurements of head rotation angles θ and associated pressure changes q can be performed. The data can be represented in matrix form as:

$$Θ z = Ψ {A}_{1} Γ \qquad ( 2 )$$

The Kronecker product principle can be applied to obtain the least squares solution for A₁. Once A₁ is known, θ_{z} at any instance can be estimated using A₁ and the different pressure measurements. Since complete 6DoF head motion is defined by 3 rotations and 3 translations (θx, θy, θz, Tx, Ty, Tz), the controller 114 can extract six calibration matrices (A₁, A₂, . . . and A₆) that can map the pressure inputs to the corresponding 6DoF head motions.

Methods of this disclosure can include calibrating the system 100. The system 100 can be calibrated inside the MRI scanner 104 or outside of the MRI scanner 104. An advantage of calibrating outside the MRI scanner 104 is that there can be no need for patient cooperation inside the scanner and no increase in actual in-scanner time since the procedure can be performed in any setting prior to the scan. Another benefit is that the calibration can be more rigorous since more data can be obtained for accurate modeling.

Pressure sensor 112 calibration can be performed on a standard patient bed with a 3D printed model of the 32 channel head coil. The patient with his or her head inside the coil model can be instructed to move the head slowly in a) left-right continuous motions, b) foot head continuous motions and c) continuous motions tracing a figure “8” with the nose. Real time (˜9600 Hz), pressure measurements and reference motion measurements of the system 100 can be performed concurrently using an optical motion tracking system. The data can be partitioned into 80% calibrating and 20% testing sets and used in the proposed model to estimate and validate the A coefficient matrices, described above.

As described previously, the system 100 can be calibrated inside the MRI scanner 104. In embodiments, in-scanner calibrations can be performed prior to all other scans during an experiment. Different imaging-based strategies can be used for pad 102 calibration. 80% of the data can be used as the calibration set to develop the model and 20% can be used as the testing set to evaluate its performance.

One in-scanner calibration can use rapid 3D scout scans with static poses. The patient can move their head to different positions and hold their pose for highly accelerated 3-5 sec 3D scout MPRAGE scans. Reference 6DoF motion parameters estimated from 3D rigid body registrations of the scout images and concurrent sensor recordings can be used to calibrate the model. The number and degree of the necessary static poses can be optimized. Data generated from calibrations performed outside the scanner can provide initial estimates for these parameters.

Another in-scanner calibration can use real-time 2D golden angle radial imaging with continuous motion. The patient can perform in-plane motions in the left-right and foot-head direction and continuous single slice golden angle (GA) radial data can be acquired in the axial and sagittal planes respectively. The GA data can be used to reconstruct dynamic images of the moving head that are time synchronized to (via pulse program triggers fed to the sensor microprocessor) sensor data, and can be used to perform the calibration. In embodiments, a ˜6 s calibration scan can yield ˜40 brain pose images at 128 profiles/image in around ˜6 s reconstruction time.

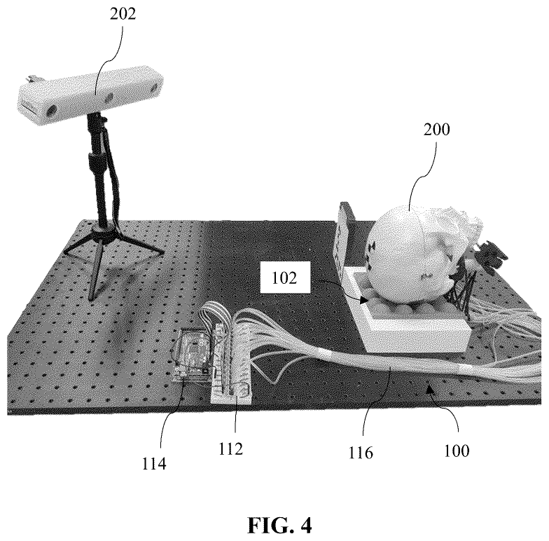

FIG. 4 shows a perspective view of an embodiment of a calibration of the system 100 outside of the MRI scanner 104. The calibration setup can include the system 100, a (e.g., plastic) skull 200 placed on the pad 102, and an optical tracker 202 for reference motion measurements. For the calibration, the skull can be manually rotated about the foot-head axis in the range of +15 degrees and 30 random recordings of rotation angle and pressure can be made. Of these, 20 data points can be used for calibrating the model, and the model can be used to predict the rotation for the 10 remaining test points. Next, the skull can be taken off the pad 102 and placed back, without taking care to replicate the exact earlier position. The skull can again be rotated to 10 different angles and data can be collected. The model for estimating the position of the skull 200 (described previously) can then be used to predict the angle after skull replacement.

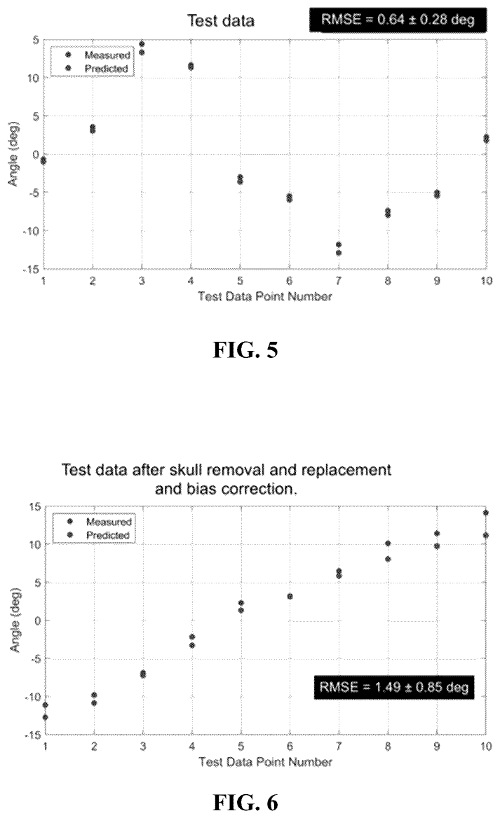

FIG. 5 shows the results of the calibration before the skull was removed. FIG. 6 shows the results of the calibration after the skull was removed. The root mean squared error (RMSE) obtained in the calibration data was 0.41±0.24 deg indicating that the model is well fitted. The RMSE for the 10 testing points prior to skull removal was only 0.64±0.28 deg (FIG. 5), indicating that the pad 102 was able to estimate the motions accurately with inside-the-scanner calibration. The rotation estimates after skull replacement initially showed an RMSE of 3.92±1.24 deg, but also showed a constant bias in the rotation estimates, likely arising from skull repositioning. After removal of the bias by simple subtraction of the 0 deg (no motion) estimate, the RMSE reduced to 1.49±0.85 deg (FIG. 6).

The system 100 and associated methods can be used to demonstrate retrospective motion correction in 2D and 3D brain imaging. A patient can be asked to perform five head motion patterns in a randomized order:
1. Keeping the head stationary. This case can assess the stability of the sensor data and validate that motion correction is not introducing artifacts when motions are minimal. This can also provide artifact-free images for reference.
2. Left-right in-axial plane motion.
3. Foot-head ‘through-axial plane’ motions consisting of slow continuous nodding and intermittent fast jerks
4. Slow continuous motions in a figure “8” pattern to test for multiaxis rotations.
5. Pseudo-random motions: the worst case in which the volunteers move their head randomly with mixed motions.
   All motions can be restricted to <±10° of rotation and <±10 mm of translation. The subjects can be trained on the motions to perform in the calibration session conducted outside the scanner prior to the scan.

Images can be then reconstructed retrospectively using a conjugate gradient optimization-based generalized SENSE algorithm that incorporates shot wise motion estimates:

$$[ \hat{x} ] = {arg min}_{x} {‖ di - {K}_{i} {FSM}_{i} x ‖}^{2} 2 \qquad ( 3 )$$

where x is the unmoved image, di is the shot-wise motion corrupted MR data, i is the shot index, K is the k-space sampling function, F is the Fourier operator, S is the unmoved coil sensitivities, M are the shot wise motion data from the sensor, and \hat{x} is the estimate of the unmoved image. To assess improvement of image quality, the normalized 3D image gradient entropy can be estimated for each reconstructed image. Mean and standard deviations (stdev) of the entropy values over all slices can be calculated and compared using statistical test, such as a Wilcoxon signed-rank test.

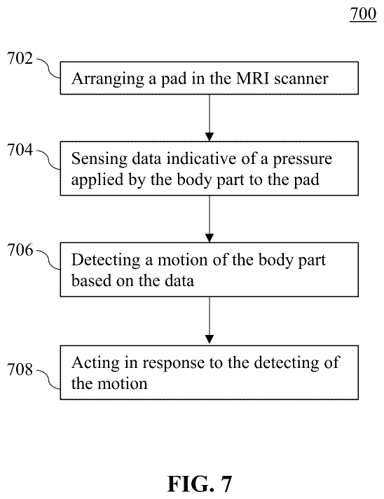

FIG. 7 shows a process 700 of correcting for motion of a body part (e.g., a head) in the MRI scanner 104. The process 700 can use any of embodiments of the system 100 described previously. The process 700 can include, at step 702, arranging the pad 102 in the MRI scanner 104, as previously described.

The process 700 can include, at step 704, sensing, via the plurality of sensors 112 that are operatively connected to the pad 102, a pressure applied by the body part to the pad 102, as previously described. As also described previously, the plurality of sensors 112 can be MRI compatible. As previously described, sensing of the pressure applied by the body part to the pad 102 can include sensing a pressure of a fluid partially contained within the pad 102.

In embodiment, the process 700 can include between steps 704 and step 706 Performing pad 102 calibration by using instructed head motions and MRI images, as previously described. The calibration can also be performed outside the MRI scanner 104, prior to the scan, as previously described.

The process 700 can include, at step 706, detecting a motion of the body part based on the data, as previously described. The detecting can occur during scanning of the body by the MRI scanner 104.

The process 700 can include, at step 708, acting in response to the detecting of the motion of the body part at step 706. In embodiments, acting in response to the detecting of the motion of the body part comprises at least one of estimating the motion of the head of the patient, automatically stopping the scanner, signaling that the motion has been detected, estimating a quantity of data corrupted by the motion, estimating a quantity of data corrupted by the motion and signaling the quantity, or providing feedback to patient.

The system 100 and associated methods do not interfere with the MRI sequence (i.e. uses non-NMR detection), can be used prospectively (i.e. allows fast motion detection and feedback to the sequence), doesn't require markers (i.e. patient is oblivious to the presence of the sensor), works for any MRI sequence (2D or 3D) and does not rely on line of sight through the head coil. The system 100 can use a subject-specific pre-calibration technique and can estimate 6 DoF head motions and can correct images retrospectively.

The system 100 and associated methods can improve temporal SNR in resting state fMRI with pad-based motion detection and data rejection of a single motion incident. The system 100 can demonstrate retrospective generalized SENSE-based correction of motion corrupted images in a range of whole brain 2D and 3D structural and functional sequences based on the standard ABCD protocol, specifically: 2D and 3D Fast Spin Echo, 2D T2*w Gradient Echo, 3D T1w Gradient Echo and 2D single-shot Echo Planar Imaging based resting state BOLD fMRI. Motion correction can be demonstrated using a quantitative metric of image quality.

The system 100 and associated methods can provide for non-NMR, pressure-based head motion tracking that can be applicable regardless of sequence type. The system 100 can sense data indicative of head motion (e.g., pressure) without using NMR signals, light, RF or head acceleration.

The system 100 and associated methods, including for example the pressure sensors 112, can be completely MR compatible and can fit inside any standard multichannel receive head coil. That is, the system 100 and associated methods do not interfere with images generated by the MRI scanner 104. The pad 102 can be easily portable from scanner to scanner and coil to coil for easy universal applicability.

The system 100 and associated methods can also include a mathematical model-based motion calibration method that can be performed on a subject specific basis outside the scanner using a 3D printed model of the head coil. Performing calibration outside the scanner can prevent any increase in scan time. Additionally, rapid, in-scanner calibration methods based on ultrafast scout scans and golden angle radial scans can be used with the system 100 and associated methods.

The cost of utilizing system 100 and associated methods can be minimal. The system 100 and associated methods can provide a scan-agnostic technique to detect head motions in real time and improve data quality either by rejecting and reacquiring data, or by allowing real time operator feedback to the patient. The system 100 and associated methods can generate detailed mapping of the pressure changes caused by head motions in an MRI scan, and how they relate to the degree and type of motion. The system 100 and associated methods can be utilized to generate novel data on the variability of pressure changes in a population of different head sizes and shapes.

The system 100 and associated methods can provide a universal, real time, calibration-free head motion correction technique in MRI.

The system 100 and associated methods can be applied to other imaging and radiation oncology modalities. In such embodiments, MRI compatibility of the sensors is not a factor and the sensor pad can be scaled up in density and sensor quality to allow for more accurate and faster motion estimations.

The system 100 and associated methods are advantageous in their independence from the MRI sequence, universal applicability, low need for patient compliance and potential for prospective application.

While certain implementations of the invention have been described, the present disclosure is not limited to these disclosed aspects. Additional modifications and improvements to the disclosure may be apparent to those skilled in the art. Moreover, the many features and advantages of the disclosure are apparent from the detailed specification, and thus, it is intended by the appended claims to cover all such features and advantages of the present disclosure which fall within the spirit and scope of the disclosure.

What is claimed is:

1. A system for detecting a motion of a head of a patient in a scanner, the system comprising:
a pad that is configured to support the head of the patient within the scanner, the pad comprising a plurality of protrusions that protrude from a surface of the pad, each of the plurality of protrusions define a chamber filed with a fluid;
a plurality of pressure sensors, each of the plurality of pressure sensors being operatively connected with a respective chamber of the plurality of protrusions, and each of the plurality of pressure sensors being configured to sense a pressure within each of the respective chambers and to generate data indictive of the pressure; and
a controller that is configured to:
receive the data indicative of the pressure within each of the respective chambers; and
detect the motion of the head of the patient based on the data indicative of the pressure within each of the respective chambers.

2. The system of claim 1, wherein when the controller detects the motion of the head of the patient during a scan of the patient by the scanner the controller is configured to at least one of estimate the motion of the head of the patient, automatically stop the scanner, signal that the motion has been detected, estimate a quantity of data corrupted by the motion, estimate a quantity of data corrupted by the motion and signal the quantity, or provide feedback to patient.

3. The system of claim 1, wherein each of the plurality of protrusions define a convex shape.

4. The system of claim 1, wherein the fluid is a gas.

5. The system of claim 1, wherein the fluid is a liquid.

6. The system of claim 1, further comprising a plurality of fluid pathways that fluidly connect each respective chamber of the plurality of protrusions with the respective pressure sensor of the plurality of pressure sensors.

7. The system of claim 6, wherein the plurality of fluid pathways each include a segment that is integral with the pad.

8. The system of claim 1, wherein the scanner is an MRI scanner, and wherein the system further comprises an MRI scanner configured to scan the head of the patient.

9. The system of claim 8, wherein the controller is further configured to:
estimate the motion of the head based on the data indicative of the pressure within each of the respective chambers; and
correct a scan of the head of the patient from the MRI scanner based on the estimate of the motion of the head.

10. The system of claim 9, wherein the controller is configured to correct the scan of the head of the patient from the MRI scanner after completion of the scan.

11. The system of claim 9, wherein the controller is configured to correct the scan of the head of the patient during the scan.

12. The system of claim 8, wherein the plurality of pressure sensors are provided outside of an interior of the MRI scanner.

13. The system of claim 8, wherein the MRI scanner comprises an MRI head coil,
wherein the MRI head coil defines a space within an interior of the MRI head coil, and
wherein the space is configured to receive the pad and the head of the patient.

14. The system of claim 1, wherein the plurality of pressure sensor are MRI compatible.

15. A method for detecting motion of a body part in a scanner, the method comprising:
arranging a pad in the scanner;
sensing, via a plurality of sensors operatively connected to the pad, a pressure applied by the body part to the pad;
detecting a motion of the body part based on the pressure; and
acting in response to the detecting of the motion of the body part.

16. The method of claim 15, wherein acting in response to the detecting of the motion of the body part comprises at least one of estimating the motion of the head of the patient, automatically stopping the scanner, signaling that the motion has been detected, estimating a quantity of data corrupted by the motion, estimating a quantity of data corrupted by the motion and signaling the quantity, or providing feedback to patient.

17. The method of claim 15, further comprising estimating, in response to detecting the motion of the body party, the motion of the body part based on the pressure and imaging the body part using the scanner and the motion estimated based on the data.

18. The method of claim 15, wherein the detecting the motion is performed during a scan of the body part by the scanner.

19. The method of claim 15, wherein the sensing of the pressure applied by the body part to the pad comprises sensing a pressure of a fluid partially contained within the pad.

20. The method of claim 15, wherein scanner is an MRI scanner, and
wherein the plurality of sensors are MRI compatible.
